(19) United States
(12) Patent Application Publication
(10) Pub. No.: US 2006/0251659 A1
(43) Pub. Date: Nov. 9, 2006

(54) Method for screening molecules that restore NOD1 activity in cells containing an NOD2 mutation that reduces or eliminates NOD1 activity

(76) Inventors: Stephen Girardin, Toronto (CA); Mihai Netea, Nijmegen (NL)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(21) Appl. No.: 11/399,477

(22) Filed: Apr. 7, 2006

Related U.S. Application Data
(60) Provisional application No. 60/670,674, filed on Apr. 13, 2005.

(51) Int. Cl. A61K 48/00; G01N 33/53; A61K 39/395

(52) U.S. Cl. 424/145.1; 514/44; 435/7.1

(57) ABSTRACT

A method for identifying a molecule that restores Nod1 activity in cells which contain a Nod2 mutation that reduces or eliminates Nod1 activity. Nod2/CARD15 is the first characterized susceptibility gene in Crohn's disease. The Nod2 1007fs (Nod2fs) frameshift mutation is the most prevalent in Crohn's disease patients. Muramyl dipeptide (MDP) from bacterial peptidoglycan is the minimal motif detected by Nod2 but not by Nod2fs. The inventors investigated the response of human peripheral blood mononuclear cells (PBMCs) from Crohn's disease patients not only to MDP, but also to several other muramyl peptides. Unexpectedly, it was observed that patients homozygous for the Nod2fs mutation were totally unresponsive to MurNAc-L-Ala-D-Glu-mesoDAP (M-Tri_{DAP}), the specific agonist of Nod1. Accordingly, Gram-negative bacterial peptidoglycan, which can be detected by both Nod1 and Nod2, was unable to stimulate cytokine secretion from Nod2fs PBMCs. While M-Tri_{DAP} acts in synergy with both LTA and LPS to induce cytokine secretion from PBMCs of healthy donors, this phenomenon is attenutated in cells from Nod2fs patients.

### BACKGROUND OF THE INVENTION

1. Field of the Invention

A method for identifying a molecules which modulates the activity of Nod pattern recognition molecules, especially which modulates Nod1 activity in subjects having an Nod2 mutation, such as Nod2fs, which reduce or eliminate Nod1 activity. The method is useful for identifying agents which modulate or restore Nod activity and thus would be useful pharmaceutical agents for treating diseases, such as Crohn's Disease associated with deficits or disruptions of Nod activity.

2. Description of the Related Art

Nod2 (also known as CARD15) is a member of the Nod family of pattern recognition molecules (PRMs) involved in peptidoglycan sensing (1, 2). While Nod2 detects a muramyl dipeptide (MDP) motif found in peptidoglycans from all classes of bacteria (3-5), Nod1 detects a diaminopimelic acid (DAP)-containing muramyl tripeptide (M-Tri_{DAP}) found primarily in Gram-negative bacterial peptidoglycan (4, 6, 7). In addition to its role as an intracellular PRM, genetic evidence has identified Nod2 as the first susceptibility gene for Crohn's disease (8, 9). Crohn's disease is an inflammatory disorder affecting the digestive tract, the etiology of which remains largely unknown. However, the recent association between the disease and Nod2 on the one hand, and between Nod2 and bacterial sensing on the other, suggests that Crohn's disease is likely a consequence of a breakdown in the tolerance to the intestinal bacterial flora. Still, it remains unclear why Nod2 dysfunction is a risk factor favoring the onset of Crohn's disease. Indeed, while Nod2fs is fully defective for peptidoglycan sensing, other Nod2 mutant proteins found in Crohn's disease patients display only minor differences in peptidoglycan detection (10, 11).

Through the identification of new important functions of Nod2, substantial progress has been made over the past few years towards understanding the link between Nod2 mutations and Crohn's disease (1, 2, 12). For example, Nod2 function has been shown to be related to intracellular bacterial killing (13), defensin activity due to its expression in Paneth cells (14, 15) as well as the induction of the anti-inflammatory cytokine IL-10 (16). Also Nod2^{-/-} mice display an increased T_{H}1 profile of cytokine responses following stimulation with Toll-like receptors (TLRs) agonists (17), which is compatible with some features of Crohn's disease.

Maeda et al. (31) describe the Nod2 mutation in Crohn's Disease and Kobayashi et al. (32) describe Nod2-dependent regulation of innate and adaptive immunity. The DNA sequences of Nod1, Nod2 and Nod2 mutants have been described. Nod1 sequences are described by J. Bertin (30); Nod2 sequences by Y. Ogura et al. (27) and those of some Nod2 mutants by J. P. Hugot et al. (8) and Y. Ogura et al. (9). Said sequences are hereby incorporated by reference.

### BRIEF SUMMARY OF THE INVENTION

The inventors have discovered the existence of an unexpected cross-talk between the Nod1 and Nod2 signaling pathways. That is, a mutation in the Nod2 gene, such as the Nod2fs mutation, also causes a defect in Nod1 function. Nod2fs is a mutation of the Nod2 gene and is associated with susceptibility to Crohn's Disease. While Nod2fs has been associated with Crohn's Disease, the resulting functional deficiency of Nod1 activity associated with the Nod2fs mutation may operate to cause or aggravate disease. Based on this discovery, methods for identifying molecules which restore Nod function, such as restoring a functional Nod1 pathway in Nod2fs patients, are disclosed.

The inventors investigated the response of primary mononuclear cells isolated from Crohn's disease patients not only to MDP, but also to several muramyl peptides or peptidoglycan agonists. Using this approach, they identified M-Tri_{LYs} and M-Tetra_{LYS}, two MDP-related muramyl peptides, as Nod2 agonists. In addition, they show that the whole peptidoglycan polymers extracted from Staphylococcus aureus and Streptococcus pneumoniae fails to induce cytokine response in PBMCs from Nod2fs patients. Zouali et al. (29) argued against a major role of Nod1 in inflammatory bowel disease (including Crohn's disease) genetic susceptibility. Thus, surprisingly it was found that M-Tri_{DAP}, the specific agonist of Nod1, also failed to induce cytokine response in PBMCs from Nod2fs patients, while it efficiently stimulated cells from either healthy donors or non Nod2 Crohn's disease patients. The importance of these discoveries is further reinforced by the observation that cells from Nod2fs patients were totally unresponsive to peptidoglycan from Helicobacter pylori, which is an efficient activator of both Nod1 and Nod2 signaling pathways. Muramyl peptides are known to induce cytokine secretion in synergy with TLR ligands.

The inventor's show that the blockage of Nod1 function is only partially overcome in PBMCs stimulated with M-Tri_{DAP} in combination with either LPS or LTA. These discoveries now provide the foundation for design of novel diagnostic methods for Crohn's disease based on detection of defects in Nod1 function and therapeutic approaches for treatment of Crohn's disease which establish functional Nod1 pathway in Nod2fs subjects.

### BRIEF DESCRIPTION OF THE DRAWINGS

FIG. 1. Detection of MDP-derived muramyl peptides by Nod2. A, schematic representation of the muramyl peptides used in this study. B, Human HEK293 epithelial cells were transfected with several muramyl peptides (MDP, M-Tri_{Lys}, Anh-M-Tri_{Lys}, M-Tetra_{LYS} and Anh-M-Tetra_{LYS}) at the following concentrations: 10 nM (white bars), 50 nM (grey bars) or 250 nM (black bars) in the presence of an expression vector for Nod2 . The activity of a NF-κB-driven luciferase reporter gene was measured, and Nod2-dependent activation of the reporter gene in the presence of muramyl peptides was reported to the one obtained without stimulation with muramyl peptides. Data show the mean±s.e.m of duplicates. Experiments were performed three times with similar results.

FIG. 2. Response to muramyl peptides of PBMCs from healthy donors (CTR), Crohn's disease patients without defects in Nod2 (Crohn's) or Crohn's disease patients homozygous for the frameshift mutation (Nod2 fs). IL-1β, TNF and IL-10 were measured from the cell culture medium. Intracellular IL-1α was measured from cell lysates. All muramyl peptides (all 50 nM) were added directly to the cell culture medium for 18 hours.

FIG. 3. Response to peptidoglycans of PBMCs from healthy donors (CTR), Crohn's disease patients without defects in Nod2 (Crohn's) or Crohn's disease patients homozygous for the frameshift mutation (Nod2fs). IL-1β, TNF and IL-10 were measured from the cell culture medium. Intracellular IL-1α was measured from cell lysates. All peptidoglycans (all at 1 μg/ml) and LPS (100 ng/ml) were added directly to the cell culture medium for 18 hours.

FIG. 4. Expression of Nod1 in PBMCs from 13 individuals by real-time PCR. Expression of Nod1 was analyzed by real-time PCR in cells from healthy donors (CTR), Crohn's disease patients without defects in Nod2 (Crohn's) or Crohn's disease patients homozygous for the frameshift mutation (Nod2 fs). Expression of Nod1 is reported to the internal control β-actin.

FIG. 5. Analysis of the synergistic activation of PBMCs by M-Tri_{DAP} plus either LPS or LTA. PBMCs from healthy donors (CTR), Crohn's disease patients without defects in Nod2 (Crohn's) or Crohn's disease patients homozygous for the frameshift mutation (Nod2 fs) were analyzed. Cells were stimulated either with M-Tri_{DAP} (MTP(DAP) on the figure), LPS or LTA alone or in combination; LPS+M-Tri_{DAP} or LTA+M-Tri_{DAP} with the agonists added simultaneously, IL-1β, TNF and IL-10 were measured from the cell culture medium. Intracellular IL-1α was measured from cell lysates, LPS (40 pg/ml), LTA (5 μg/ml) and M-Tri_{DAP} (50 nM) were added directly to the cell culture medium for 18 hours.

### DETAILED DESCRIPTION OF THE INVENTION

Results and Discussion

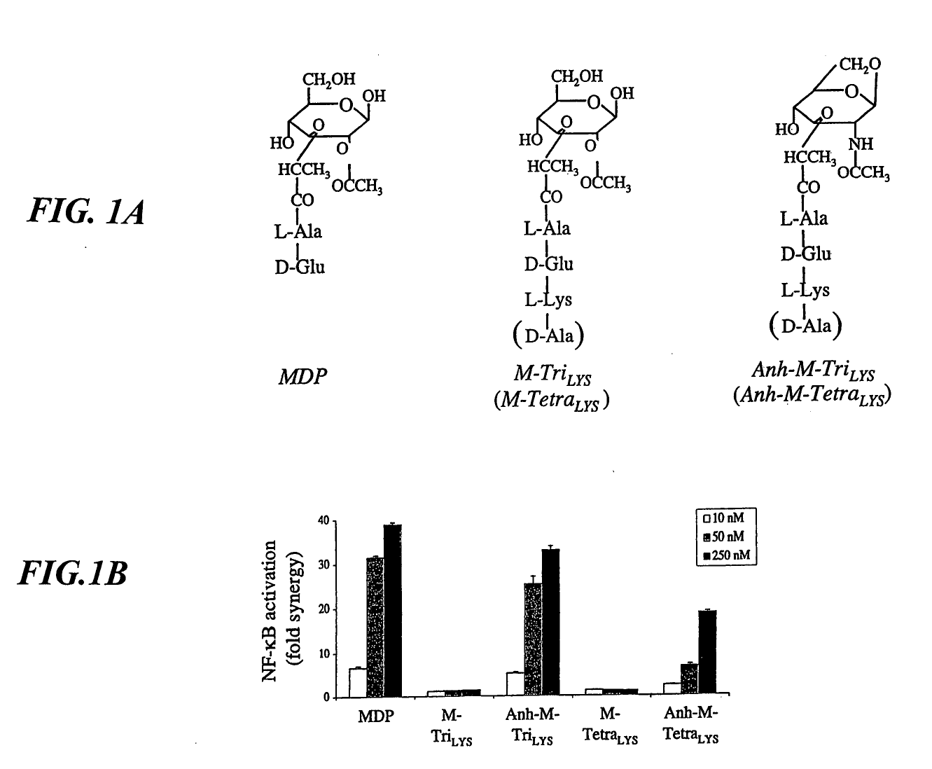

In search for MDP-derived muramyl peptides that could stimulate the Nod2 signaling pathway, the inventors generated several molecules differing in the length of their peptidic moiety, including M-Tri_{Lys} and M-Tetra_{Lys} (FIG. 1A). These molecules were then tested for their ability to activate Nod2 using co-transfection assays in HEK293T epithelial cells and measuring NF-κB activity as a read-out (FIG. 1B). Using such tests, it was found that the activation of Nod2 was maximal with the addition of 10 pmoles of MDP per milliliter of culture medium (leading to a concentration of 10 nM). Here, a larger range of muramyl peptide concentration was used (10 nM up to 250 nM) to allow for the identification of even weak inducers of the Nod2 pathway. Through this approach, it was found that MDP and M-Tri_{Lys} activated Nod2 with similar efficiency, while M-Tetra_{Lys} represented a poor agonist (FIG. 1B). These results are consistent with previous observations showing that the length of the muramyl peptide stem peptide is a key requirement for induction of Nod2 (4).

One goal was to use MDP-derived muramyl peptides to stimulate primary human peripheral blood mononuclear cells (PBMCs) (see below). Thus, the inventors searched for other MDP-derived molecules that could represent the optimal negative controls for M-Tri_{Lys} and M-Tetra_{Lys} agonists.

By taking advantage of a previous observation that the sugar moiety of muramyl peptides also plays a key role for optimal activation of Nod2 (4), modified forms of M-Tri_{Lys} and M-Tetra_{Lys} were generated in which the MurNAc moiety is dehydrated to form anhydro-muramyl peptides (see FIG. 1A). It was observed that this subtle modification was sufficient to abolish stimulation of Nod2 (FIG. 1B). Therefore, anhydro-M-Tri_{Lys}, and anhydro-M-Tetra_{Lys} could be used as control inactive muramyl peptides for M-Tri_{Lys}, and M-Tetra_{Lys}, respectively. Finally, M-Tri_{DAP}, the specific muramyl peptide agonist of Nod1, failed to activate Nod2, even at the highest dose (250 nM) used.

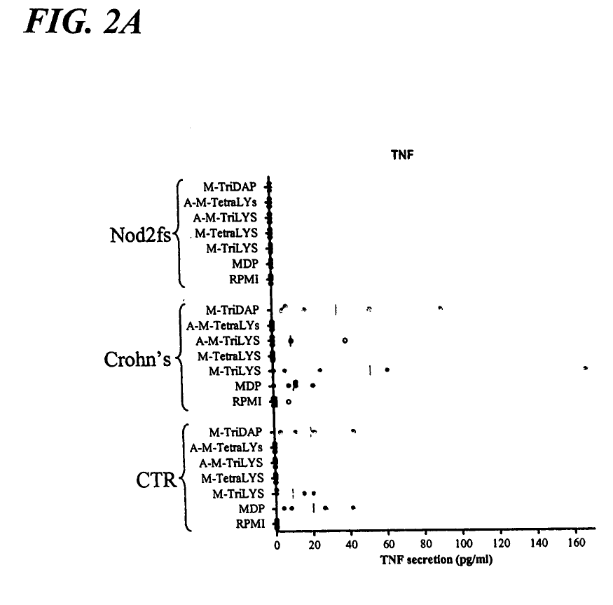

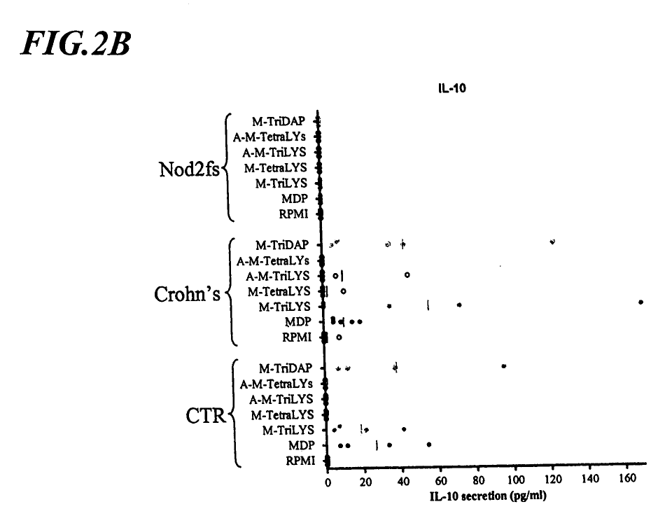

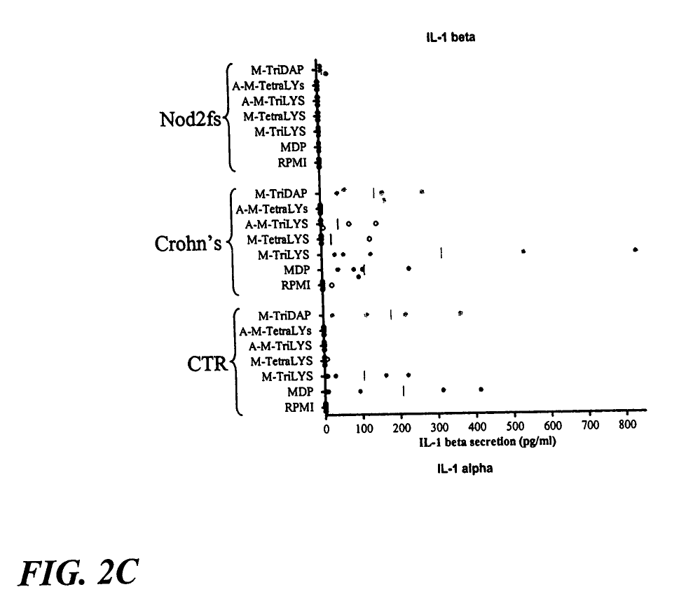

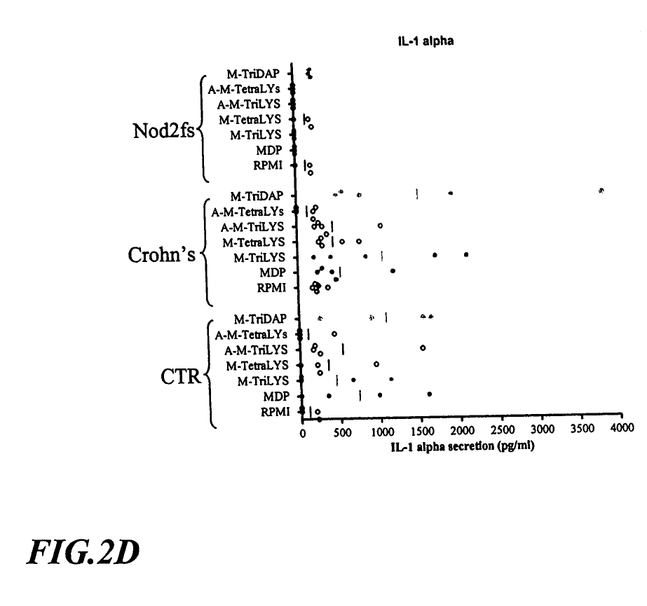

The six muramyl peptides characterized above (MDP, M-Tri_{Lys}, M-Tetra_{Lys}, anhydro-M-Tri_{Lys}, anhydro-M-Tetra_{Lys} and M-Tri_{DAP}) were used to stimulate PBMCs obtained from human blood. PBMCs from three groups of individuals were collected: healthy donors (CTR), Crohn's disease patients without Nod2 mutations (Crohn) and Crohn's disease patients carrying homozygous Nod2fs frameshift mutation (Nod2fs). Muramyl peptides were directly added to the culture medium at a final concentration of 50 nM, and supernatants were collected following overnight stimulation. IL-1β, IL-10 and TNFα were measured in the supernatant, while intracellular IL-1α was measured from cell lysates (FIG. 2). First, the results identified MDP and M-Tri_{Lys}, as potent activators of human PBMC responses and the inventors confirmed that the detection of these muramyl peptides depends upon Nod2 since Nod2fs cells were not stimulated by MDP and M-Tri_{Lys} (FIG. 2).

In addition, it was observed that M-Tetra_{Lys} was a poor inducer of Nod2 in vitro was reinforced by findings that this agonist only marginally induced cytokine secretion from human PBMCs, and that this effect was further blunted in cells from Nod2fs patients (FIG. 2). Importantly, the results show that Nod2-independent Crohn's disease patients still reacted to MDP, M-Tri_{Lys} and M-Tetra_{Lys}, thus demonstrating that the inability of cells from Nod2fs patients to detect these agonists resulted from their Nod2 mutation and was not an indirect consequence of the disease. Consequently, this observation also suggests that defects in muramyl peptide sensing are not the sole cause of Crohn's disease development.

Second, the conclusion that sensing of M-Tri_{Lys} and M-Tetra_{Lys} in PBMCs from healthy donors and non-Nod2 Crohn's disease patients depends on Nod2 is reinforced by the observation that anhydro-M-Tri_{Lys} and anhydro-M-Tetra_{Lys} failed to stimulate these cells, which is in agreement with the results obtained in HEK293T cells (see FIG. 1B). Finally, the inventors aimed to use M-Tri_{DAP} in order to stimulate PBMCs in a Nod1-dependent but Nod2-independent manner. It must be noted that, while the profile of cytokine response of macrophages to MDP has been studied extensively in the past (18), such information is largely missing for M-Tri_{DAP} mainly because this muramyl peptide is difficult to synthesize or isolate from bacteria. Here, it was shown that M-Tri_{DAP} was globally as potent as MDP in inducing cytokines in human PBMCs from healthy donors or non-Nod2 Crohn's disease patients (FIG. 2). Strikingly, it was observed that M-Tri_{DAP} was unable to stimulate PBMCs from Nod2fs patients.

This result was unexpected since M-Tri_{DAP} is a specific activator of Nod1, but not of Nod2. This lack of response to M-Tri_{DAP} was found for all the cytokines that were tested, suggesting that the defect must lie far upstream in the Nod1-dependent signaling pathway, likely at the level of the detection by the Nod1 sensing system. Therefore, these results identify an unexpected link between Nod2 mutations and the Nod1 signaling pathway.

Muramyl peptides are naturally occurring degradation products of peptidoglycan which are useful tools to study precisely the involvement of signaling pathways dependent upon the specific activation of Nod1 or Nod2. However, in physiological situations, macrophages would likely encounter the presence of both intact peptidoglycan polymers together with muramyl peptides. Therefore, the inventors aimed to investigate the response of PBMCs from the same individuals to peptidoglycans from H. pylori, S. pneumoniae and S. aureus.

The inventors decided to use peptidoglycan from H. pylori since it is the prototype of Gram-negative bacterial peptidoglycan (DAP-type peptidoglycan) and it is relatively easy to purify. Similarly, peptidoglycan from S. pneumoniae was chosen since it represents a classical peptidoglycan (Lys-type peptidoglycan) from Gram-positive bacteria. Finally, peptidoglycan from S. aureus was also included in this study since it is widely studied; however, because of the extremely high degree of peptidic cross-linking found in this peptidoglycan, its structure is less representative of Gram-positive bacterial peptidoglycan than that of S. pneumoniae.

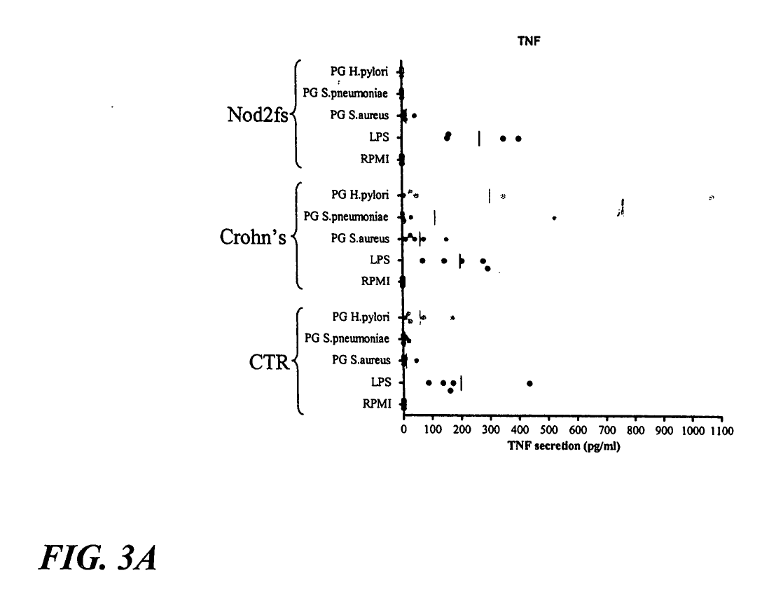

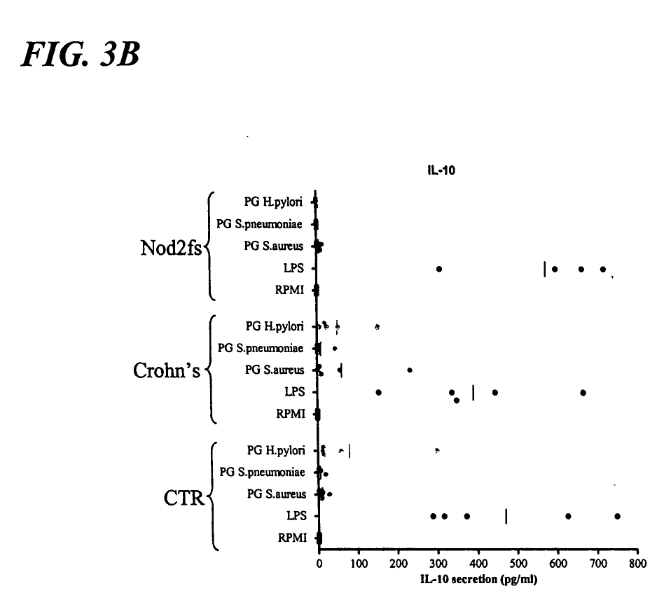

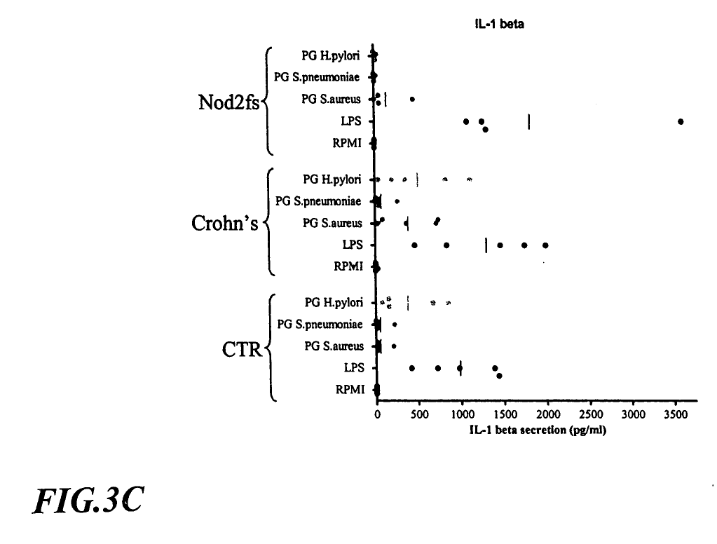

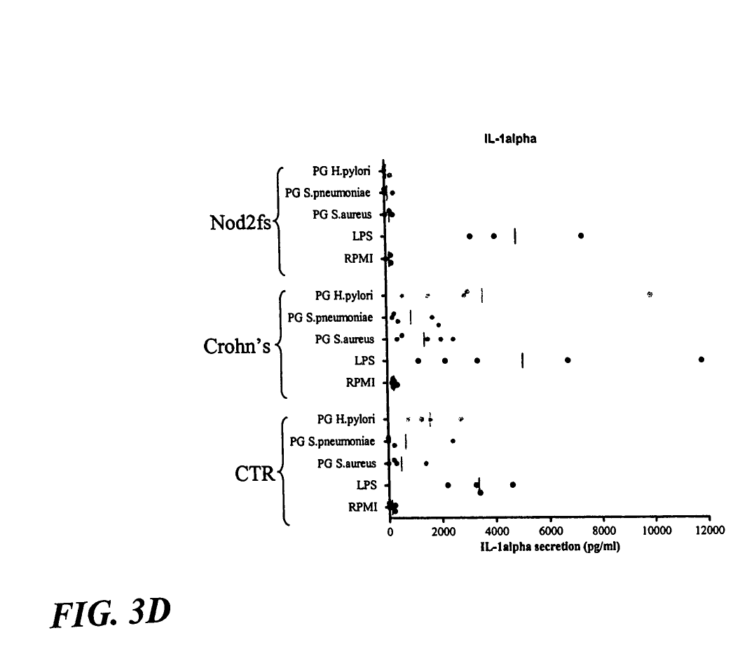

For such studies, the level of purification of the peptidoglycan polymer is a crucial feature. Several quality control tests were performed along the purification steps to ensure that other cell wall contaminants are excluded, such as lipopolysaccharide (LPS), lipoproteins or lipoteichoic acid (LTA). To this end, the absence of LPS contamination was assessed by the Limulus Amoebocyte Lysate test, showing that purified peptidoglycans contained less than 4 pg LPS/ml sample. To address the difficult question of contamination by lipoproteins or LTA, the inventors took advantage of their recent observation that contaminant-free peptidoglycans fail to stimulate thioglycholate-induced peritoneal macrophages from mice (19). The inventors' purified peptidoglycans failed to induce the secretion of TNFA or IL-6 from peritoneal mouse macrophages, showing that only traces amounts, if any, of lipoproteins or LTA contaminants were present in their peptidoglycan preparations. These peptidoglycan preparations were then added (each at 10 μg/ml) to the human PBMCs from the same individuals as described above, and cytokines were measured after over-night stimulation (FIG. 3). As a control, cells were also stimulated with LPS (1 ng/ml). First, by analyzing the cytokine response of cells from the healthy donors, the inventors noticed that peptidoglycans are strong activators of human PBMCs, which is in sharp contrast with the response of mouse peritoneal macrophages (see above). The reason of this discrepancy remains unknown, but it strongly correlates with the blunted response of mouse macrophages to muramyl peptides (S. E. Girardin, unpublished results). Second, the inventors observed that cells from non-Nod2 Crohn's disease patients also responded to peptidoglycans as well (or even slightly more, depending on the cytokines) as the healthy donors (FIG. 3). Finally, the inventors found that PBMCs from the Nod2fs group of patients were totally unresponsive to the three peptidoglycans used in this study, regardless of the cytokine analyzed. Importantly, these cells were still fully responsive to LPS stimulation, thus demonstrating that Nod2fs PBMCs did not display a global unresponsiveness to any stimulation. Again, these results are in perfect agreement with the data from cells stimulated with muramyl peptides (see FIG. 2).

The observation that PBMCs from Nod2fs patients are unresponsive to Gram-positive bacterial peptidoglycans allows one to draw some important conclusions. First, this result shows that Nod2 is key sensor of Gram-positive bacterial peptidoglycan. Second, this observation suggests that, within the peptidoglycan polymer, the MDP and M-Tri_{Lys}, motifs are the key structures that drive the host's response through their detection by Nod2. Third, the inventors' assumption that these peptidoglycans are free of bacterial contaminants is reinforced by this result, since unpurified peptidoglycans would have induced a TLR-driven response. In the case of the Gram-negative bacterial peptidoglycan, it was anticipated that, in the macrophages from Nod2fs patients, the defective Nod2 sensing could be compensated by the activation of the Nod1 signaling pathway. Indeed, unlike Gram-positive bacterial peptidoglycan, Gram-negative bacterial peptidoglycan is able to stimulate both Nod1 and Nod2. The lack of Nod1-dependent signalization in Nod2fs cells (FIG. 3) again suggests that a functional Nod2 signaling pathway is required for the Nod1-driven signalization to take place. This result confirms and extends the conclusions from the study of muramyl peptide stimulation of PBMCs (see FIG. 2). Taken together, it can be concluded that any peptidoglycan sensing (dependent upon Nod2, Nod1, or any uncharacterized peptidoglycan sensor) is abrogated in PBMCs from Nod2fs patients. This defect is not found in non-Nod2 Crohn's disease patients, therefore suggesting that if lack of peptidoglycan sensing contributes to the onset of Crohn's disease, the pathology can also arise from other causes.

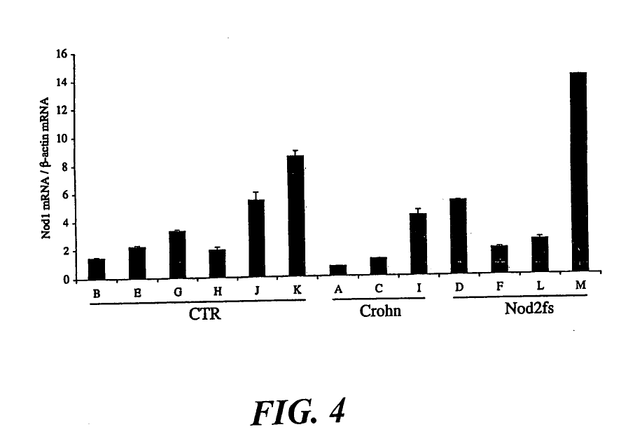

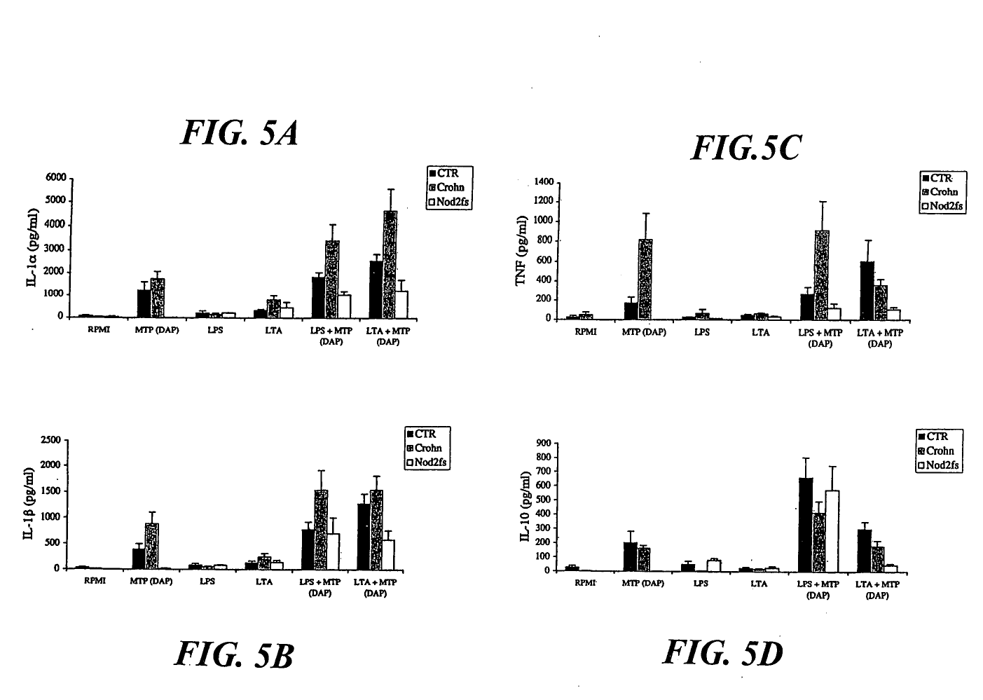

In an attempt to better understand the origin of the defective Nod1-dependent signaling in cells from Nod2fs patients, the inventors first investigated if Nod1 expression was decreased in Nod2fs PBMCs. Nod1 expression was analyzed by real-tin PCR on 13 individuals (6 “CTR”, 3 “Crohn's” and 4 “Nod2fs”). Even though expression of Nod1 was found quite variable among individuals, no correlation could be observed between expression levels of Nod1 and the three groups analyzed (FIG. 4). Therefore, the lack of Nod1-dependent response in Nod2fs PBMCs can not be accounted for by a defect in Nod1 expression in these cells. Next, the inventors analyzed if the lack of response of Nod2fs cells to M-Tri_{DAP} was still observed in the case of co-stimulation with other agonists. Indeed, it is well characterized that muramyl peptides act in synergy with TLR agonists to induce cytokine secretion from PBMCs (20). Therefore, the inventors stimulated PBMCs from individuals in their three groups (CTR, Crohn, Nod2fs) with M-Tri_{DAP}, LPS (TLR4 agonist) or LTA (TLR2 agonist) either alone or in combination (M Tri_{DAP}+LPS or M-Tri_{DAP}+LTA). For the three groups of individuals and for all the cytokines studied, it was observed that M-Tri_{DAP} could function in synergy with LPS or LTA to potentiate cytokine secretion (FIG. 5). However, it was noticed that in PBMCs from Nod2fs patients, the observed synergy between M-Tri_{DAP} and LTA or LPS remained lower than in CTR or Crohn group of individuals. The most striking defect was a lack of synergy between M-Tri_{DAP} and LTA for IL-10 secretion in PBMCs from Nod2fs patients, while a synergy between M-Tri_{DAP} and LPS was observed (FIG. 5D). Together, these results suggest that even though M-Tri_{DAP} does not directly induce cytokine secretion in Nod2fs PBMCs, the blockage can be partially overcome in the case of co-stimulation with TLR ligands.

These results clearly demonstrate that PBMCs from Nod2fs patients are unable to induce cytokine secretion following stimulation with M-Tri_{DAP}, the specific agonist of Nod1. The molecular basis of this defect, however, remains unclear. In light of the inventors' experiments, it can be excluded that the defective Nod1-dependent signaling pathway arises from an altered expression of Nod1 itself (see FIG. 4). It is possible that Nod2fs interacts with and titrates out a co-factor crucial for Nod1 function. Alternatively, cells from Nod2fs patients may constitutively express an unknown factor which would lock the Nod1 signaling pathway. The defective Nod1 function in PBMCs from Nod2fs patients was, at least in part, overcome when cells were co-stimulated with TLR ligands, such as LPS or LTA (see FIG. 5). This observation strongly suggests that in cells expressing functional TLRs, the defective Nod1 pathway may not have a crucial impact on the etiology of Crohn's disease. However, this defect could prove of critical importance in epithelial cells lining mucosal surfaces. Indeed, these cells are permanently in contact with microbes and microbial products, and therefore down-regulation of TLR function represents a common mechanism to avoid constitutive inflammation due to the microbial flora (21). Accordingly, using ex vivo experiments, the inventors have been able to show that intestinal epithelial cells detect nonflagellated bacteria exclusively through Nod1 (6). As a consequence, it can be envisioned that defective function of Nod1 in intestinal epithelial cells from Nod2fs patients may participate in the establishment of Crohn's disease.

The results presented here can be applied to the design of new therapeutic treatments for Crohn's disease. Nod2 1007fs mutation represents one third to half of the Nod2 mutations found in Crohn's disease patients. Thus, in this group of patients, a therapeutic approach that restores a functional Nod1 signaling would reverse underlying defects caused by the Nod2 mutation. Indeed, up until now, the idea of targeting Nod1 pathway in Crohn's disease patients was not envisioned in this way, since it was assumed that Nod1 remained fully functional. Restoring a functional Nod1 pathway in Nod2fs cells would have the important advantage to restore partial homeostasis of the intestinal mucosa vis-à-vis the microbial environment. Therefore, since Crohn's disease can be associated with a breakdown in the tolerance to the intestinal bacterial flora, such tolerance could be restored through the Nod1-dependent sensing of Gram-negative bacterial components of the microbial environment. Because such therapy would rely on a fine balance defined by the host itself, it would be less aggressive than other treatments, such as those acting to reduce the inflammation induced by the disease.

### EXAMPLES

### Example 1

### Analysis of Nod1 Activity in Cells having Nod2fs Mutation

Experimental Procedures

Preparation of Highly Purified Peptidoglycans from Gram-Negative and Gram-Positive Bacteria

Bacterial strains used to prepare PGN are the following: Helicobacter pylori 26695; Staphylococcus aureus COL (from Olivier Chesneau, Institut Pasteur); Streptococcus pneumoniae R800. The PGN purification procedures were exactly as previously described (6, 19). Purity of samples was assessed by HPLC amino acid and saccharide analysis after HCl hydrolysis. Also, each PGN preparation was tested or the absence of LPS contamination using the Limulus Ameobocyte Lysate assay as previously described (19). The absence of TLR2-detected contaminants (lipoproteins or lipoteichoic acids) was tested on thioglycholate elicited mouse peritoneal macrophages from either C57B16 or TLR2% mice as previously described (19).

Preparation of Muropeptides

DAP- and Lys-containing UDP-MurNAc-peptides were prepared as described previously (4 22). M-Tetra_{Lys}, M-Tri_{Lys} and M-Tri_{DAP} were generated by mild acid hydrolysis (0.1 M HCl, 10 min at 100° C.) of the corresponding UDP-MurNAc-peptides. Replacement of meso-DAP by L-Lys in the peptidoglycan of E. coli was obtained by overexpression in the latter species of the murE gene from Staphylococcus aureus encoding UDP-MurNAc-L-Ala-L-D-GIu; L-Lys adding enzyme (23). Cells were harvested before cell lysis occurs and their peptidoglycan was extracted and purified as previously described (24). In these conditions, about 50% of the DAP residues at the third position of the peptides were shown to be replaced by L-Lys. This peptidoglycan preparation was digested by SltY lytic transglycosylase in a reaction mature (1 ml) consisting in 300 mM sodium acetate buffer, pH 4.5, 1 mg of PG (briefly sonicated for homogenization), and 100 μg purified SltY enzyme (25). After overnight incubation at 37° C., the reaction was stopped by adding 500 μl of 50 mM sodium phosphate buffer, pH 4.45 (HPLC eluent A) and 2 μl phosphoric acid. The two main monomer products, Anh-GM-Tetra_{DAP} and Anh-GM Tetra_{Lys}, were purified by HPLC on a column of nucleosyl 5C₁₈ (4,6×250 mm, Alltech). Elution was performed at 0.6 ml/min with buffer A, using a gradient of methanol from 0 to 25% in 180 min. Detection was at 215 nm. The retention times of these two compounds were 67 min and 80 min, respectively. They were further purified and desalted using a second HPLC step on the same column using this time 0.1% trifluoroacetic acid and a gradient of methanol for elution. Their purity and composition was confirmed by amino acid and hexosamine analysis after acid hydrolysis of samples (6 M HCl, 16 h at 95° C.), using a Hitachi L8800 analyzer, as well as by MALDI-TOF mass spectrometry. Anh-M-Tetra_{Lys} was obtained by treatment of Anh-GM-Tetra_{Lys} with E. coli NagZ β N-acetylglucosaminidase. The reaction mixture (200 μl) contained 20 mM HEPES buffer, pH 7.4, 50 mM NaCl, 0.5 mM substrate, and 20 hg of purified NagZ enzyme (25), Anh-GM-Tri_{Lys} and Anh-M-Tri_{Lys} were generated by treatment of the corresponding tetrapeptide compounds with E. coli LdcA L,D-carboxypeptidase. The reaction mixture (200 μl) contained 50 mM Tris-HCl buffer, pH 8.0, 0.5 mM substrate, and 20 μg purified LdcA enzyme (25). In all cases, incubation was for overnight at 37° C. and products were purified by HPLC and their identity confirmed by the above described procedures.

Cell Lines and Reagents

HEK293T cells were cultured in DMEM containing 10% fetal calf serum. Prior to transfection, HEK293T cells were seeded into 24 well plates at a density of 1×10⁵ cells/ml as described previously (26), MDP LD was from Calbiochem and reported to be 98% pure by TLC.

Expression Plasmids and Transient Transfections

The expression plasmid for Nod2 has been previously described (27). The NF-κB luciferase reporter plasmid was from Stratagene. Transfections were carried out in BEK293T cells as previously described (26).

NF-κB Activation Essays

Studies examining the synergistic activation of NF-κB by muramyl peptides in cells over expressing Nod2 were carried out as originally described by Inohara et al. (28). Briefly, HEK293T cells were transfected overnight with 10 ng of Nod2 plus 75 ng of NF-κB luciferase reporter plasmid. At the same time, muramyl peptides were added to cell culture medium and the synergistic NF-κB-dependent luciferase activation was then measured following 24 h of co-incubation. NF-κB-dependent luciferase assays were performed in duplicate and data represent at least 3 independent experiments. Data show mean±SEM.

Genotyping of NOD2 Variants

Blood was collected from 74 patients with Crohn's disease and 10 healthy volunteers, PCR amplification of NOD2 gene fragments containing the polymorphic site 3020insC was performed in 50 μl reaction volumes containing 100-200 ng genomic DNA, as previously described (16). The 3020insC polymorphism was analyzed by Genescan analysis on an ABI Prism 3100 Genetic Analyzer according to the protocol of the manufacturer (Applied Biosystems, Nieuwerkerk a/d IJssel, The Netherlands).

Four patients with Crohn's disease were found homozygous for the 3020insC mutation, and they were further investigated in the cytokine studies. As control groups, five patients with Crohn's disease heterozygous for the 3020 insC NOD2 mutation, five patients with Crohn's disease bearing the wild type allele, and five healthy volunteers homozygous for the wild-type NOD2 allele were included. The cells isolated from the four groups of patients were isolated and tested at two separate occasions. The study was approved b the Ethical Committee of the Radboud University, Nijmegen, the Netherlands.

Isolation of Mononuclear Cells and Stimulation of Cytokine Production

After informed consent, venous blood was drawn from the cubital vein of patients and healthy volunteers into three 10 ml EDTA tubes (Monoject, s-Hertogenbosch, The Netherlands). The PBMCs fraction was obtained by density centrifugation of blood diluted 1:1 in pyrogen-free saline over Ficoll-Paque (Pharmacia Biotech AB, Uppsala, Sweden). Cells were washed twice in saline and suspended in culture medium (RPMI 1640 M) supplemented with gentamicin 10 mg/ml, L-glutamine 10 mM and pyruvate 10 mM. The cells were counted in a Coulter counter (Coulter Electronics, Mijdrecht, The Netherlands) and the number was adjusted to 5×10⁶ cells/ml. 5×10⁵ PBMC in a 100 μl volume were added to round-bottom 96-wells plates (Greiner, Alphen a/d Rijn, The Netherlands) and incubated with either 100 μl of culture medium (negative control), or the various stimuli: 50 nM of the various muropeptide preparations, 10 μg/ml of the purified peptidoglycans 1 ng/ml highly purified E. coli LPS (strain 055 :B5), 5 μg/ml of artificially-synthesized LTA (kindly provided by dr. Corinna Hermann, Konstanz University, Germany), or a combination of stimuli as described in the Results section.

Cytokine Measurements

For detection of cytokine concentrations in the supernatants, BioPlex 100 system (BIO-RAD, Hercules, Calif., U.S.A.) was used. The kits were used as indicated by manufacturer and the sensitivity for all cytokines was <20 pg/ml.

Quantification of Nod 1 mRNA using Real-Time PCR

Total RNA was isolated from cells using Rneasy kits (Macherey Nagel, Hoerdt, France) according to the manufacturer's instructions. RNA quantification was performed using spectrophotometry. After treatment at 37° C. for 30 min with 20-50 units of RNase-free DNase I (Roche Diagnostics Corporation, Indianapolis, USA) were used to synthesize single-stranded cDNA. Nod1 mRNA was quantified using SYBR green Master Mix (Applera, Courtaboeuf, France) with specific human oligonucleotides (sense: GTAAAGGTGCTA AGCGAAGA, anti-sense; TCTGATTCTGGATAAGCCAT) in a GeneAmp Abiprism 7000 (Applera, Courtaboeuf, France). In each assay, calibrated and no-template controls were included. Each sample was run in duplicate, SYBR green dye intensity was analyzed using the Abiprism 7000 SDS software (Applera, Courtaboeuf, France). All results were normalized to the β-actin, an unaffected housekeeping gene.

Statistical Analysis

The human experiments were performed in triplicate with blood obtained from patients and volunteers. The differences between groups were analyzed by Mann-Whitney U test, and where appropriate by Kruskal-Wallis ANOVA test. The level of significance between groups was set at p<0.05. The data are given as means±SD.

### Example 2

### Screening Method for Identifying Compounds which Restore Nod1 Activity in Cells having an Nod2 Mutation

Isolation of PBMCs:

Human PBMCs (peripheral blood mononuclear cells) from a Nod2fs patient are isolated. 10-20 ml of blood from these subjects is obtained and yields about 5-7×10⁶ PBMCs. Cells are isolated and seeded in 96 well plates at 10⁵ cells/well. From a single subject an average of 60 wells can be obtained.

Stimulation of PBMCs.

The objective of the screening method is to identify a compound which allows Nod2fs cells to detect a Nod1 specific agonist such as M-Tri_{DAP}, the specific ligand of Nod 1. Thus, in each well cells are incubated overnight with 100 nM M-Tri_{DAP} or with a control without M-Tri_{DAP}. The following day, the amount of stimulation induced by exposure to M-Tri_{DAP} is determined by measuring cytokine secretion in the cell supernatants obtained from the overnight incubations. Levels of different cytokines may be measured, including IL-1β, IL-10 and/or TNFα Cytokine levels are determined by conventional ELISA procedures. In addition to M-Tri_{DAP} each individual well is stimulated with a candidate molecule of interest or with an appropriate control. Conventional methods for detecting or measuring cytokines are known. Some of those methods are described by Example 1 above.

Technical Aspects of the Test.

The screening method is based on a gain-of-function (i.e., restoring the sensitivity of the Nod2fs macrophages to the Nod1 specific ligand) rather than a loss-of-function. Thus, it is not necessary to investigate the possible toxicity of each test compound at this stage of the screening. Moreover, the gain-of-function approach allows the assay to test hundreds of test molecules at a single experimental point. For example, to screen a bank of 100,000 different test molecules, the molecules may be pooled in to groups of 170 molecules, in which case the blood obtained from about 10 patients would be adequate to screen the bank (170×60×10). Then, using a dichotomic approach, a group of interest (170 molecules) can be redivided to identify one or a smaller group of candidate molecules.

Modifications and Other Embodiments

Various modifications and variations of the described methods and products as well as the concept of the invention will be apparent to those skilled in the art without departing from the scope and spirit of the invention. Although the invention has been described in connection with specific preferred embodiments, it should be understood that the invention is not intended to be limited to such specific embodiments. Various modifications of the described modes for carrying out the invention which are obvious to those skilled in the immunological, microbiological, molecular biological, medical, biological, chemical or pharmacological arts or related fields are intended to be within the scope of the following claims.

### INCORPORATION BY REFERENCE

The documents cited above in conjunction with the description of particular methods or products are incorporated by reference for the purpose of describing these particular methods or products.

### REFERENCES

(1) Girardin, S. E., J. P. Hugot, and P. J. Sansonetti, 2003, Lessons from Nod2 studies: towards a link between Crohn's disease and bacterial sensing. Trends Immunol 24:652.
(2) Inohara, N., M. Chamaillard, C. McDonald, and G. Nunez. 2004. NOD-LRR Proteins: Role in Host-Microbial Interactions and Inflammatory Disease. Annu Rev Biochem.
(3) Girardin, S. E., I. G. Boneca, J. Viala, M. Chamaillard, A. Labigne, G. Thomas, D. J. Philpott, and P. J. Sansonetti. 2003. Nod2 is a general sensor of peptidoglycan through muramyl dipeptide (MDP) detection. J Biol Chem 278:8869.
(4) Girardin, S. E., L. H. Travassos, M. Herve, D. Blanot, I. G. Boneca, D. J. Philpott, P. J. Sansonetti, and D. Mengin-Lecreulx. 2003. Peptidoglycan molecular requirements allowing detection by Nod1 and Nod2. J Biol Chem 278:41702.
(5) Inohara, N., Y. Ogura, A. Fontalba, O. Gutierrez, F. Pons, J. Crespo, K. Fukase, S. Inamura, S. Kusumoto, M. Hashimoto, S. J. Foster, A. P. Moran, J. L. Femandez-Luna, and G. Nuriez. 2003. Host recognition of bacterial muramyl dipeptide mediated through NOD2. Implications for Crohn's disease. J Biol Chem 278:5509.
(6) Girardin, S. E., I. G. Boneca, L. A. Cameiro, A. Antignac, M. Jehanno, J. Viala, K. Tedin, M. K. Taha, A. Labigne, U. Zahringer, A. J. Coyle, P. S. DiStefano, J. Bertin, P. J. Sansonetti, and D. J. Philpott. 2003. Nod1 detects a unique muropeptide from gram-negative bacterial peptidoglycan. Science 300:1584.
(7) Chamaillard, M., M. Hashimoto, Y. Horie, J. Masumoto, S. Qiu, L. Saab, Y. Ogura, A. (7) Kaasaki, K. Pukase, S. Kusumoto, M. A. Valvano, S. J. Foster, T. W. Mak, G. Nunez, and N. Inohara. 2003. An essential role for NODI in host recognition of bacterial peptidoglycan containing diaminopimelic acid, Nat Immunol 4.702.
(8) Hugot, J. P., M. Chamaillard, H. Zouali, S. Lesage, J. P. Cezard, J. Belaiche, S. Almer, C. Tysk, C. A. O'Morain, M. Gassull, V, Binder, Y. Finkel, A. Cortot, R. Modigliani, P. Laurent- Puig, C. Gower-Rousseau, J. Mac, J. F, Colombel, M. Sahbatou, and G. Thomas. 2001. Association of NOD2 leucine-rich repeat variants with susceptibility to Crohn's disease. Nature 411:599.
(9) Ogura, Y., D. K. Bonen, N. Inohara, D. L. Nicolae, F. F. Chen, R. Ramos, H. Britton, T. Moran, R. Karaliuskas, R. H, Duerr, J. P. Achkar, S. R. Brant, T. M. Bayless, B. S. Kirschner, S. B. Hanauer, G. Nunez, and J. H. Cho. 2001. A frameshift mutation in NOD2 associated with susceptibility to Crohn's disease. Nature 411:603.
(10) Chamaillard, M., D. Philpott, S. E. Girardin, H. Zouali, S. Lesage, F. Chareyre, T. H. Bui, M. Giovannini, U. Zaehringer, V. Penard-Lacronique, P. J. Sansonetti, J. P. Hugot, and G. Thomas. 2003. Gene-environment interaction modulated by allelic heterogeneity in inflammatory diseases. Proc Natl Acad Sci USA 100:3455.
(11) Bonen, D. K., Y. Ogura, D. L. Nicolae, N. Inohara, L. Saab, T. Tanabe, F. F. Chen, S. J. Foster, R. H. Duerr, S. R. Brant, J. H. Cho, and G. Nunez. 2003. Crohn's disease-associated NOD2 variants share a signaling defect in response to lipopolysaccharide and peptidoglycan. Gastroenterology 124:140.
(12) Rogler, G. 2004. Update in inflammatory bowel disease pathogenesis. Curr Opin Gastroenterol 20.311.
(13) Hisamatsu, T., M. Suzuki, H. C, Reinecker, W. J. Nadeau, B. A. McCormick, and D. K. Podolsky. 2003. CARD15/NOD2 functions as an antibacterial factor in human intestinal epithelial cells, Gastroenterology 124:993.
(14) Ogura, Y., S. Lala, W. Xin, E. Smith, T. A. Dowds, F. F. Chen, E. Zimmermann, M. Tretiakova, J. H. Cho, J. Hart, J. K. Greenson, S. Keshav, and G. Nunez. 2003. Expression of NOD2 in Paneth cells: a possible link to Crohn's ileitis. Gut 52:1591.
(15) Wehkamp, J., J. Harder, M. Weichenthal, M. Schwab, E. Schaffeler, M. Schlee, K. R. Herrliager, A. Stallmach, F. Noack, P. Fritz, J. M. Schroder, C. L. Bevins, K. Fellermann, and (E. F, Stange. 2004. NOD2 (CARD15) mutations in Crohn's disease are associated with diminished mucosal alpha-defensin expression. Gut 53:1658.
(16) Netea, M. G., B. J. Kullberg, D. J. de Jong, B. Franke, T. Sprong, T. H. Naber, J. P. Drenth, and J. W. Van der Meer. 2004. NOD2 mediates anti-inflammatory signals induced by TLR2 ligands: implications for Crohn's disease, Eur J Immunol 34:2052.
(17) Watanabe, T., A. Kitani, P. J. Murray, and W. Strober. 2004. NOD2 is a negative regulator of Toll-like receptor 2-mediated T helper type 1 responses. Nat Immunol 5:800.
(18) Pabst, M. J., S. Beranova-Giorgianni, and J. M. Krueger. 1999. Effects of muramyl peptides on macrophages, monokines, and sleep. Neuroimmunomodulation 6:261.
(19) Travassos, L. H., S. E. Girardin, D. J. Philpott, D. Blanot, M. A. Nahori, C. Werts, and I. G. Boneca. 2004. Toll-like receptor 2-dependent bacterial sensing does not occur via peptidoglycan recognition. EMBO Rep 5:1000.
(20) Girardin, S. E., and D. J. Philpott, 2004. Mini-review; the role of peptidoglycan recognition in innate immunity, Eur J Immunol 34:1777.
(21) Philpott, D. J., S. E. Girardin, and P. J. Sansonetti. 2001. Innate immune responses of epithelial cells following infection with bacterial pathogens. Curr Opin Immunol 13:410.
(22) Flouret, B., D. Mengin-Lecreulx, and J. van Heijenoort. 1981. Reverse-phase high- pressure liquid chromatography of uridine diphosphate N-acetylmuramyl peptide precursors of bacterial cell wall peptidoglycan. Anal Biochem 114:59.
(23) Mengin-Lecreulx, D., T. Falla, D. Blanot, J. van Heijenoort, D. J. Adams, and I. Chopra 1999. Expression of the Staphylococcus aureus UDP-N-acetylmuramoyl-L-alanyl-D-glutamate; L-lysine ligase in Escherichia coli and effects on peptidoglycan biosynthesis and cell growth. J Bacteriol 181:5909.
(24) Mengin-Lecreulx, D., and J. van Heijenoort. 1985. Effect of growth conditions on peptidoglycan content and cytoplasmic steps of its biosynthesis in Escherichia coli. J Bacteriol 163:208.
(25) Stenbak, C. R., J. H. Ryu, F. Leulier, S. Pili-Floury, C. Parquet, M. Herve, C. Chaput, I. G. Boneca, W. J. Lee, B. Lemaitre, and D. Mengin-Lecreuix 2004. Peptidoglycan molecular requirements allowing detection by the Drosophila immune deficiency pathway. J Immunol 173:7339.
(26) Girardin, S. E., R. Toumebize, M. Mavris, A. L. Page, X. Li, G. R. Stark, J, Bertin, P. S. DiStefano, M. Yaniv, P. J, Sansonetti, and D. J. Philpott. 2001. CARD4/Nod1 mediates NF-kappaB and JNK activation by invasive Shigella flexneri. EMBO Rep 2:736.
(27) Ogura, Y., N. Inohara, A. Benito, F. F. Chen, S. Yamaoka, and G. Nunez. 2001. Nod2 , a Nod1/Apaf-1 family member that is restricted to monocytes and activates NF-kappaB. J Biol Chem 276:4812.
(28) Inohara, N., Y. Ogura, F. F. Chen, A. Muto, and G. Nunez. 2001. Human Nod1 confers responsiveness to bacterial lipopolysaccharides. J Biol Chem 276:2551.
(29) Zouali, H., Lesage, S., Merlin, F., Cezard, J-P, and the EPWG-IBD group. CARD4/NOD1 is not involved in inflammatory bowel disease. 2003. Gut 52:71.
(30) Bertin, J., Nir, W-J, Fischer, C. M., Tayber, O. V., Erranda, P. R., Grant, J. R., Keilty, J. J., Gosseline, M. L., Robson, K. E., Wong, G. H. W., Glucksmann, M. A., and DiStefano, P. S. 1999. Human CARD4 protein is a novel CED-4/Apaf-1 cell death family member that activates NF-κB. J. Biol. Chem. 274, 12955.
(31) Maeda, S., Hsu L-C, Liu, H., Bankston, L. A., limura, M., Kagnoff, M. F., Eckmann, L. and Karin, M. 2005. Mod2 mutation in Crohn's disease potentiates NfκB activity and IL-1β processing. Science 307:734.
(32) Kobayashi, K. S., Chamaillard, M., Ogura, Y., Henegarlu, O., Inohara, N., Nufiez, G., Flavell, R. A. Nod2 -dependent regulation of innate and adaptive immunity in the intestinal tract. 2005. Science 307:731.

1. A method for identifying a molecule that restores Nod1 function, comprising:
contacting a cell having Nod1 and which has a Nod2 mutation which reduces or
eliminates at least one Nod1 function, with at least one candidate molecule, or
expressing said candidate molecule in said cell, and
determining the amount of functional Nod1 activity of in said cell after exposure to he at least one candidate molecule.

2. The method of claim 1, comprising contacting a cell having a Nod2 mutation that decreases or eliminates Nod1 activity, with a Nod1 specific agonist.

3. The method of claim 1, comprising contacting a cell having the Nod2fs mutation that decreases or eliminates Nod1 activity, with a Nod1 specific agonist.

4. The method of claim 1, comprising contacting a cell that is homozygous for the Nod2fs mutation that decreases or eliminates Nod1 activity, with a Nod1 specific agonist.

5. The method of claim 1, wherein said cell is obtained from a subject having Crohn's disease.

6. The method of claim 1, wherein said cell is a macrophage.

7. The method of claim 1, comprising:
contacting the cell having a Nod2 mutation that decreases or eliminates Nod1 activity, with M-Tri_{DAP}, and
determining the amount of cytokine release as an indicator of the degree of Nod1 stimulation or inhibition provided by the candidate molecule.

8. The method of claim 1, wherein the level of IL-1β, IL-10 and/or TNFα is measured as an indicator of the degree of Nod 1 stimulation or inhibition provided by the candidate molecule.

9. The method of claim 1 wherein the release into the supernatant of IL-1β, IL-10 and TNF-α is determined.

10. The method of claim 1 wherein the intracellular concentration of Il-1α is determined.

11. The method of claim 1, wherein the response of the cell to an Nod1 agonist is determined.

12. The method of claim 1 wherein said candidate molecule is an organic molecule having a molecular mass of 2,500 Da or less.

13. The method of claim 1, wherein said candidate molecule is a peptide or a protein.

14. The method of claim 1, wherein said candidate molecule is an antibody.

15. The method of claim 1, wherein said candidate molecule is a nucleic acid.

16. The method of claim 1, further comprising determining the ability of the candidate molecule to bind to at least a portion of the Nod1 , Nod2 , or Nod2 mutant molecule.

17. A method for restoring Nod1 activity in a cell having an Nod2 mutation that reduces or eliminates Nod1 activity by comprising: inserting wild-type Nod2 or wild-type Nod2 gene into said cell.

18. The method of claim 17, wherein the Nod2 mutation is Nod2fs.

19. The method of claim 17, wherein the wild-type Nod2 gene is inserted into the cell on a vector.

20. A method for restoring Nod1 activity in a cell having a mutant Nod2 gene which comprises contains a mutation of the wild-type Nod2 sequence that reduces or eliminates Nod1 activity, comprising:
repairing the mutant Nod2 gene by inserting a polynucleotide which is complementary to the polynucleotide sequence of the mutant Nod2 gene, except at the site of the error where it has the sequence of the wild-type Nod2 gene.

21. A method for classifying a subject having Crohn's Disease or a digestive tract disorder, or liable to have Crohn's Disease or a digestive disorder, comprising:
determining whether said subject is responsive to Nod1 and Nod2 agonists, Nod1 agonists but not Nod2 agonists, Nod2 agonists but not Nod1 agonists, or unresponsive to both Nod1 and Nod2 agonists.

22. A method for restoring tolerance to the intestinal bacterial flora in a subject having a Nod2 mutation wherein said method comprises:
administering to said subject a molecule that restores Nod1 function.

23. The method of claim 22, wherein said molecule is identified by:
contacting a cell having Nod1 and which has an Nod2 mutation which reduces or
eliminates at least one Nod1 function, with at least one candidate molecule, or
expressing said candidate molecule in said cell, and
determining the amount of functional Nod1 or Nod2 activity, or both, of in said cell after exposure to the at least one candidate molecule.

24. The method of claim 22, wherein said molecule is wild-type Nod2 or Nod2 gene.

25. The method of claim 22, wherein the method further comprises administering to said subject a molecule which is a TLR4 agonist or a TLR2 agonist which functions in synergy with M-Tri_{DAP}.

26. The method of claim 22, wherein said subject has Crohn's Disease.

27. The method of claim 26, wherein the subject is a Nod2fs subject.

28. The method of claim 22, wherein the subject is unresponsive to both Nod1 and Nod2 agonists, but said subject expresses Nod1.
